(19) United States
(12) Patent Application Publication
(10) Pub. No.: US 2021/0371877 A1
(43) Pub. Date: Dec. 2, 2021

(54) ADENO-ASSOCIATED VIRUS (AAV) VECTOR LIPID NANOPARTICLE COMPOSITIONS AND METHODS OF USE

(71) Applicant: SPARK THERAPEUTICS, INC., Philadelphia, PA (US)

(72) Inventors: Andrew William SHAW, Harleysville, PA (US); Xavier ANGUELA, Philadelphia, PA (US)

(73) Assignee: SPARK THERAPEUTICS, INC., Philadelphia, PA (US)

(21) Appl. No.: 15/733,246

(22) PCT Filed: Dec. 18, 2018
(86) PCT No.: PCT/US2018/066308

§ 371 Date: Jun. 16, 2020

Related U.S. Application Data
(60) Provisional application No. 62/607,257, filed on Dec. 18, 2017.

(51) Int. Cl. C12N 15/86; A61K 9/08; A61K 9/107; A61K 9/51; C12N 15/88

(52) U.S. Cl. CPC C12N 15/86; A61K 9/08; C12N 2750/14143; A61K 9/5123; C12N 15/88; A61K 9/107

(57) ABSTRACT

A composition includes an adeno-associated vims (AAV) vector in a lipid nanoparticle (LNP). The AAV vector can include a heterologous nucleic acid sequence, optionally an inverted terminal repeat (ITR) positioned 5′ of the heterologous nucleic acid sequence and an ITR positioned 3′ of the heterologous nucleic acid sequence. The AAV vector can further include expression control elements (e.g., a promoter and/or enhancer), and intron, and/or or a polyadenylation signal.

### RELATED APPLICATIONS

This patent application claims the benefit of priority to U.S. Provisional Patent Application No. 62/607,257, filed Dec. 18, 2017. The entire content of the foregoing application is incorporated herein by reference, including all text, tables and sequence listing.

### INTRODUCTION

The invention relates to compositions and methods to deliver AAV vectors for therapeutic use. In particular, the invention relates to lipid nanoparticle (LNP) encapsulated or coated AAV vectors which inhibit, suppress, reduce or prevent AAV vector immunogenicity and subsequent development of an immune response against the AAV vector in vivo.

### SUMMARY

In some aspects, embodiments herein relate to compositions comprising an adeno-associated virus (AAV) vector in a lipid nanoparticle (LNP), the AAV vector comprising a heterologous nucleic acid sequence and an inverted terminal repeat (ITR) positioned 5′ of the heterologous nucleic acid sequence and an ITR positioned 3′ of the heterologous nucleic acid sequence.

in some embodiments, the LNP comprises a cationic lipid. In some aspects, the cationic lipid comprises an amino lipid. In some aspects, the cationic lipid is present in an amount from about 10% by weight of the LNP to about 75% by weight of the LNP.

In some embodiments, the LNP further comprises a sterol. In some aspects, the sterol is present in an amount from about 5% by weight of the LNP to about 50% by weight of the LNP.

In some embodiments, the LNP comprises a neutral lipid. In some aspects, the neutral lipid is present in an amount from about 0.1% by weight of the LNP to about 15% by weight of the LNP.

In some embodiments, the LNP comprises polyethylene glycol (PEG) or a PEG-modified lipid. In some aspects, the PEG or PEG-modified lipid is present in an amount from about 0.5% by weight of the LNP to about 10% by weight of the LNP.

In some embodiments, the LNP has a size in a range from about 10 nm to 500 nm.

In some embodiments, the LNP has a size in a range from about 50 nm to 200 nm.

In some embodiments, the LNP has a size in of about 100 nm.

In some embodiments, the LNP further comprises a cell targeting or cell penetrating moiety.

In some embodiments, the heterologous nucleic acid sequence comprises or encodes a blood coagulation Factor.

In some embodiments, the heterologous nucleic acid sequence comprises or encodes a Factor VII, VIII, IX, X, XI, V, XII, II, von Willebrand factor, vitamin K epoxide reductase C1, or gamma-carboxylase.

In some embodiments, the heterologous nucleic acid sequence comprises or encodes GAA (acid alpha-glucosidase); ATP7B (copper transporting ATPase2); alpha galactosidase; ASS1 (arginosuccinate synthase); beta-glucocerebrosidase; beta-hexosaminidase A; SERPING1 (C1 protease inhibitor); glucose-6-phosphatase; erythropoietin (EPO; interferon-alpha; interferon-beta; interferon-gamma; an interleukin (IL); any one of Interleukins 1-36 (IL-1 through IL-36); interleukin (IL) receptor; a chemokine; chemokine (C-X-C motif) ligand 5 (CXCL5); granulocyte-colony stimulating factor (G-CSF); granulocyte-macrophage colony stimulating factor (GM-CSF); macrophage colony stimulating factor (M-CSF); keratinocyte growth factor (KGF); monocyte chemoattractant protein-1 (MCP-1); tumor necrosis factor (TNF); a tumor necrosis factor (TNF) receptor; alpha-1 antitrypsin; alpha-L-iduronidase; ornithine transcarbamoylase; phenylalanine hydroxylase (PAH); phenylalanine ammonia-lyase (PAL); lipoprotein lipase; an apolipoprotein; low-density lipoprotein receptor (LDL-R); albumin; lecithin cholesterol acyltransferase (LCAT); carbamoyl synthetase I; argininosuccinate synthetase; argininosuccinate lyase; arginase; fumarylacetoacetate hydrolase; porphobilinogen deaminase; cystathionine beta-synthase; branched chain ketoacid decarboxylase; isovaleryl-CoA dehydrogenase; propionyl CoA carboxylase; methylmalonyl-CoA mutase; glutaryl CoA dehydrogenase; insulin; pyruvate carboxylase; hepatic phosphorylase; phosphorylase kinase; glycine decarboxylase; H-protein, T-protein, cystic fibrosis transmembrane regulator (CFTR); dystrophin; microdystrophin; CT GaINAc transferase (Galgt2); or survival motor neuron (SMN).

In some embodiments, a composition comprising an AAV vector in a LNP is made by method. In one aspect, a method includes mixing an AAV vector with a LNP.

In some embodiments, a method of making a composition includes coating a vessel with one or more lipids; and adding the AAV vector to the lipid coated vessel.

In some embodiments, a method of making a composition includes coating a vessel with the AAV vector; and adding the LNP or one or more lipids to the AAV coated vessel.

In some embodiments, the AAV vector or LNP is in a liquid.

In some embodiments, the AAV vector or LNP comprises an emulsion or is in a solution.

In some embodiments, the LNP liquid or solution is substantially dried prior to adding the AAV vector or LNP to the coated vessel.

In some embodiments, a method of making a composition includes solubilizing one or more lipid components in an organic solvent to provide a lipid solution; adding the lipid solution to a vessel; removing the organic solvent; adding an AAV vector in aqueous solution to the vessel; and forming LNPs by agitating the vessel by vortexing or sonication.

In certain aspects, the removing step is carried out by heating, vacuum, passing dry air or gas into the vessel or a combination thereof while rotating the vessel.

In certain aspects, the LNPs are sorted to a homogeneous size by extrusion through one or more membranes.

In some embodiments, a method of making a composition includes solubilizing one or more lipids in an organic solvent; solubilizing an AAV vector in an aqueous buffer; and mixing the solubilized lipids and the solubilized AAV vector to form LNPs encapsulating the AAV vector.

In some embodiments, a method of making a composition includes solubilizing one or more lipids in an aqueous buffer with a detergent; adding AAV vector to the solubilized lipids; and removing the detergent by dialysis to form LNPs encapsulating the AAV vector.

In some embodiments, a method of making a composition includes solubilizing one or more lipids in an organic solvent; and adding the solubilized one or more lipids dropwise to an AAV vector in an aqueous buffer with mixing to form LNPs encapsulating the AAV vector.

### DETAILED DESCRIPTION

In various embodiments, there are provided compositions comprising an adeno-associated viral (AAV) vector and a lipid nanoparticle (LNP). As used herein, “lipid nanoparticle” or “LNP” refers to a lipid-based vesicle useful for delivery of AAV and having dimensions on the nanoscale, i.e., from about 10 nm to about 1000 nm, or from about 50 to about 500 nm, or from about 75 to about 127 nm. Without being bound by theory, the LNP is believed to provide the AAV vector with partial or complete shielding from the immune system. In one embodiment, shielding allows delivery of the AAV vector to a tissue or cell while avoiding inducing a substantial immune response against the AAV vector in vivo (e.g., in a subject such as a human). Shielding may also allow repeated administration of AAV vectors without inducing a substantial immune response against the AAV vector in vivo (e.g., in a subject such as a human). Shielding may also improve or increase AAV vector delivery efficiency in vivo (e.g., in a subject such as a human) at initial AAV vector administration as well as after repeated AAV vector administration.

Accordingly, the invention provides compositions that allow delivery of the AAV vector to a cell or tissue while suppressing, inhibiting, reducing or preventing AVV vector immunogenicity (e.g., the ability to induce a humoral/antibody and/or cell-mediated immune response) thereby avoiding or preventing inducing a substantial immune response in vivo and/or improving or increasing AAV vector half-life in vitro or in vivo.

In various embodiments, preparations of LNP encapsulated AAV vector include AAV vector that is not LNP encapsulated or only partially LNP encapsulated. The non- or partially LNP encapsulated AAV vector can, if desired, be removed/separated from fully LNP encapsulated AAV vector to improve the amount, proportion or concentration of fully LNP encapsulated AAV vector. Removal of non- or partially LNP encapsulated AAV vector in a preparation of encapsulated AAV vector-LNP can be achieved by passage of the preparation through an affinity matrix (e.g., column) comprising anti-AAV antibodies. The flow-through containing fully LNP encapsulated AAV vector can be collected and subsequently used to deliver a heterologous nucleic acid to cells, tissues and organs.

LNP encapsulated AAV vector may be used in the treatment of a disease or disorder including, but not limited to, “hemostasis” or blood clotting disorders such as hemophilia A, hemophilia A patients with inhibitory antibodies, hemophilia B, deficiencies in coagulation Factors VII, VIII, IX, X, XI, V, XII, II, von Willebrand factor, combined FV/FVIII deficiency, thalassemia, vitamin K epoxide reductase C1 deficiency, gamma-carboxylase deficiency; anemia, bleeding associated with trauma, injury, thrombosis, thrombocytopenia, stroke, coagulopathy, disseminated intravascular coagulation (DIC); over-anticoagulation associated with heparin, low molecular weight heparin, pentasaccharide, warfarin, small molecule antithrombotics (i.e., FXa inhibitors); and platelet disorders such as, Bernard Soulier syndrome, Glanzmann thrombasthenia, and storage pool deficiency.

LNP encapsulated AAV vector can be used in the treatment of a disease or disorder including, but not limited to, treatment of lysosomal storage diseases. LNP encapsulated AAV vector can also be used in the treatment of a disease or disorder including, but not limited to, Pompe disease; Wilson's disease; Fabry's disease; Citrullinemia Type 1; Gaucher disease Type 1; Tay Sachs disease; Hereditary Angioedema; glycogen storage disease type I (GSDI); anemia; various immune disorders, viral infections and cancer; various inflammatory diseases or immuno-deficiencies; immune disorders such as Crohn's disease; various human inflammatory diseases; epithelial tissue damage; recurrent miscarriage; HIV-related complications; insulin resistance; emphysema; chronic obstructive pulmonary disease (COPD); Mucopolysaccharidosis I (MPS I); ornithine transcarbamoylase (OTC) deficiency; Phenylketonuria (PKU); lipoprotein lipase deficiency; apolipoprotein (Apo) A-I deficiency; Familial hypercholesterolemia (FH); Hypoalbuminemia; cystic fibrosis; and muscular dystrophy.

In certain embodiments, the LNP encapsulated AAV vectors of the invention are used to treat a subject having a disease that affects or originates in the central nervous system (CNS). In certain aspects, the disease is a neurodegenerative disease. In certain aspects, the CNS or neurodegenerative disease is Alzheimer's disease, Huntington's disease, ALS, hereditary spastic hemiplegia, primary lateral sclerosis, spinal muscular atrophy, Kennedy's disease, a polyglutamine repeat disease, or Parkinson's disease. In certain aspects, the CNS or neurodegenerative disease is a polyglutamine repeat disease. In certain aspects, the polyglutamine repeat disease is a spinocerebellar ataxia (SCA1, SCA2, SCA3, SCA6, SCA7, or SCA17).

LNP encapsulated AAV vector vectors can be used to provide a protein to a subject where there is an insufficient amount of the protein or a deficiency in a functional gene product (protein), or to provide an inhibitory nucleic acid or protein to a subject who produces an aberrant, partially functional or non-functional gene product (protein) which can lead to disease. Accordingly, subjects appropriate for treatment include those having or at risk of producing an insufficient amount or having a deficiency in a functional gene product (protein), or produce an aberrant, partially functional or non-functional gene product (protein), which can lead to disease. Subjects appropriate for treatment in accordance with the invention also include those having or at risk of producing an aberrant, or defective (mutant) gene product (protein) that leads to a disease such that reducing amounts, expression or function of the aberrant, or defective (mutant) gene product (protein) would lead to treatment of the disease or reduce one or more symptoms or ameliorate the disease. Target subjects therefore include subjects that have such defects regardless of the disease type, timing or degree of onset, progression, severity, frequency, or type or duration of symptoms.

Suitable subjects include mammals, such as humans, as well as non-human mammals. The term “subject” refers to an animal, typically a mammal, such as humans, non-human primates (apes, gibbons, gorillas, chimpanzees, orangutans, macaques), a domestic animal (dogs and cats), a farm animal (poultry such as chickens and ducks, horses, cows, goats, sheep, pigs), and experimental animals (mouse, rat, rabbit, guinea pig). Human subjects include fetal, neonatal, infant, juvenile and adult subjects. Subjects include animal disease models, for example, mouse and other animal models of blood clotting diseases and others known to those of skill in the art.

LNP encapsulated AAV vector vectors can be delivered and administered by any route. Exemplary routes include systemically, regionally or locally, or by any route, for example, by injection, infusion, orally (e.g., ingestion or inhalation), or topically (e.g., transdermally). Such delivery and administration include intravenously, intramuscularly, intraperitoneally, intradermally, subcutaneously, intracavity, intracranially, transdermally (topical), parenterally, e.g. transmucosally or rectally. Exemplary administration and delivery routes include intravenous (i.v.), intraperitoneal (i.p.), intraarterial, intramuscular, parenteral, subcutaneous, intra-pleural, topical, dermal, intradermal, transdermal, parenterally, e.g. transmucosal, intra-cranial, intra-spinal, oral (alimentary), mucosal, respiration, intranasal, intubation, intrapulmonary, intrapulmonary instillation, buccal, sublingual, intravascular, intrathecal, intracavity, iontophoretic, intraocular, ophthalmic, optical, intraglandular, intraorgan, intralymphatic.

The LNP can be combined with additional components. Non limiting components include polyethylene glycol (PEG) and sterols. Accordingly, LNP can be a PEG-modified LNP or a sterol-modified LNP. Furthermore, LNP can be a PEG-modified and a sterol-modified LNP. The LNPs, combined with additional components, can be the same or separate LNPs. In other words, the same LNP can be PEG modified and sterol modified or, alternatively, a first LNP can be PEG modified and a second LNP can be sterol modified. Optionally, the first and second modified LNPs can be combined.

As a general matter, it is believed that LNP encapsulated AAV vector delivery is not dependent upon serotype tropism. Thus, such LNP encapsulated AAV vectors can be used to target cells, tissues and organs to which the AAV vector serotype would not normally exhibit strong tropism for.

In various embodiments, as a multi-component construct, the LNP surface may also include other functional moieties. Such moieties include cell targeting or cell penetrating molecules that have tropism for or target particular tissue(s) and/or cell(s). Non-limiting examples are antibodies, cell surface receptor ligands, cell penetrating peptides (e.g., such as HIV tat), etc. Liver and hepatocytes are a particular organ and cell to target with cell targeting and cell penetrating molecules.

LNP encapsulated AAV vector can be incorporated into pharmaceutical compositions, e.g., a pharmaceutically acceptable carrier or excipient. Such pharmaceutical compositions are useful for, among other things, administration and delivery of LNP encapsulated AAV vector to a subject in vivo or ex vivo.

As used herein the term “pharmaceutically acceptable” and “physiologically acceptable” mean a biologically acceptable formulation, gaseous, liquid or solid, or mixture thereof, which is suitable for one or more routes of administration, in vivo delivery or contact. A “pharmaceutically acceptable” or “physiologically acceptable” composition is a material that is not biologically or otherwise undesirable, e.g., the material may be administered to a subject without causing substantial undesirable biological effects. Thus, such a pharmaceutical composition may be used, for example in administering a LNP encapsulated AAV vector to a subject.

Such compositions include solvents (aqueous or non-aqueous), solutions (aqueous or non-aqueous), emulsions (e.g., oil-in-water or water-in-oil), suspensions, syrups, elixirs, dispersion and suspension media, coatings, isotonic and absorption promoting or delaying agents, compatible with pharmaceutical administration or in vivo contact or delivery. Aqueous and non-aqueous solvents, solutions and suspensions may include suspending agents and thickening agents.

The term “vector” refers to small carrier nucleic acid molecule, a plasmid, virus (e.g., AAV), or other vehicle that can be manipulated by insertion or incorporation of a nucleic acid. Vectors can be used for genetic manipulation (i.e., “cloning vectors”), to introduce/transfer polynucleotides into cells and/or organs, and to transcribe or translate the inserted polynucleotide in cells. An “expression vector” is a vector that contains a gene or nucleic acid sequence with the necessary regulatory regions needed for expression in a host cell. A vector nucleic acid sequence generally contains at least an origin of replication for propagation in a cell and optionally additional elements, such as a heterologous nucleic acid sequence, expression control element (e.g., a promoter, enhancer), intron, inverted terminal repeat(s) (ITRs), optional selectable marker, polyadenylation signal.

An AAV vector is derived from adeno-associated virus. AAV vectors are useful as gene therapy vectors as they can penetrate cells and introduce nucleic acid/genetic material so that the nucleic acid/genetic material may be stably maintained in cells. In addition, these viruses can introduce nucleic acid/genetic material into specific sites, for example, such as a specific site on chromosome 19. Because AAV is not associated with pathogenic disease in humans, AAV vectors are able to deliver heterologous nucleic acid sequences (e.g., that encode therapeutic proteins and agents) to human patients without causing substantial AAV pathogenesis or disease.

The term “recombinant,” as a modifier of a vector, such as a recombinant AAV (rAAV) vector, as well as a modifier of sequences such as recombinant polynucleotides and polypeptides, means that the compositions have been manipulated (i.e., engineered) in a fashion that generally does not occur in nature. A particular example of a recombinant AAV vector would be where a nucleic acid that is not normally present in the wild-type AAV genome (heterologous sequence) is inserted within the viral genome. An example of which would be where a nucleic acid (e.g., gene) encoding a therapeutic protein or polypeptide sequence is cloned into a vector, with or without 5′, 3′ and/or intron regions normally associated with the gene, within the AAV genome. Although the term “recombinant” is not always used herein in reference to an AAV vector, as well as sequences such as polynucleotides, recombinant forms including AAV vectors, polynucleotides, etc., are expressly included in spite of any such omission.

A “rAAV vector” is derived from the wild type genome of AAV by using molecular methods to remove all or a part of the wild type AAV genome, and replacing with a non-native (heterologous) nucleic acid, such as a nucleic acid encoding a therapeutic protein or polypeptide sequence. Typically, for a rAAV vector one or both inverted terminal repeat (ITR) sequences of AAV genome are retained. A rAAV is distinguished from an AAV genome since all or a part of the AAV genome has been replaced with a non-native sequence with respect to the AAV genomic nucleic acid, such as with a heterologous nucleic acid encoding a therapeutic protein or polypeptide sequence. Incorporation of a non-native (heterologous) sequence therefore defines the AAV vector as a “recombinant” AAV vector, which can be referred to as a “rAAV vector.”

A recombinant AAV vector sequence can be packaged, referred to herein as a “particle,” for subsequent infection (transduction) of a cell, ex vivo, in vitro or in vivo. Where a recombinant vector sequence is encapsidated or packaged into an AAV particle, the particle can also be referred to as a “rAAV” or “rAAV particle” or “rAAV virion.” Such rAAV, rAAV particles and rAAV virions include proteins that encapsidate or package the vector genome. Particular examples include in the case of AAV, capsid proteins.

A “vector genome” or conveniently abbreviated as “vg” refers to the portion of the recombinant plasmid sequence that is ultimately packaged or encapsidated to form a rAAV particle. In cases where recombinant plasmids are used to construct or manufacture recombinant AAV vectors, the AAV vector genome does not include the portion of the “plasmid” that does not correspond to the vector genome sequence of the recombinant plasmid. This non vector genome portion of the recombinant plasmid is referred to as the “plasmid backbone,” which is important for cloning and amplification of the plasmid, a process that is needed for propagation and recombinant AAV vector production, but is not itself packaged or encapsidated into rAAV particles. Thus, a “vector genome” refers to the nucleic acid that is packaged or encapsidated by rAAV.

“AAV helper functions” refer to AAV-derived coding sequences (proteins) which can be expressed to provide AAV gene products and AAV vectors that, in turn, function in trans for productive AAV replication and packaging. Thus, AAV helper functions include both of the major AAV open reading frames (ORFs), rep and cap. The Rep expression products have been shown to possess many functions, including, among others: recognition, binding and nicking of the AAV origin of DNA replication; DNA helicase activity; and modulation of transcription from AAV (or other heterologous) promoters. The Cap expression products (capsids) supply necessary packaging functions. AAV helper functions are used to complement AAV functions in trans that are missing from AAV vector genomes.

An “AAV helper construct” refers generally to a nucleic acid sequence that includes nucleotide sequences providing AAV functions deleted from an AAV vector which is to be used to produce a transducing AVV vector for delivery of a nucleic acid sequence of interest, by way of gene therapy to a subject, for example. AAV helper constructs are commonly used to provide transient expression of AAV rep and/or cap genes to complement missing AAV functions that are necessary for AAV vector replication and encapsidation. Helper constructs generally lack AAV ITRs and can neither replicate nor package themselves. AAV helper constructs can be in the form of a plasmid, phage, transposon, cosmid, virus, or virion. A number of AAV helper constructs have been described, such as plasmids pAAV/Ad and pIM29+45 which encode both Rep and Cap expression products (See, e.g., Samulski et al. (1989) J. Virol. 63:3822-3828; and McCarty et al. (1991) J. Virol. 65:2936-2945). A number of other vectors have been described which encode Rep and/or Cap expression products (See, e.g., U.S. Pat. Nos. 5,139,941 and 6,376,237).

The term “accessory functions” refers to non-AAV derived viral and/or cellular functions upon which AAV is dependent for replication. The term includes proteins and RNAs that are required in AAV replication, including moieties involved in activation of AAV gene transcription, stage specific AAV mRNA splicing, AAV DNA replication, synthesis of Cap expression products and AAV capsid packaging. Viral-based accessory functions can be derived from any of the known helper viruses such as adenovirus, herpesvirus (other than herpes simplex virus type-1) and vaccinia virus.

An “accessory function vector” refers generally to a nucleic acid molecule that includes polynucleotide sequences providing accessory functions. Such sequences can be on an accessory function vector, and transfected into a suitable host cell. The accessory function vector is capable of supporting rAAV virion production in the host cell. Accessory function vectors can be in the form of a plasmid, phage, transposon or cosmid. In addition, the full-complement of adenovirus genes are not required for accessory functions. For example, adenovirus mutants incapable of DNA replication and late gene synthesis have been reported to be permissive for AAV replication (Ito et al., (1970) J. Gen. Virol. 9:243; Ishibashi et al., (1971) Virology 45:317). Similarly, mutants within E2B and E3 regions have been shown to support AAV replication, indicating that the E2B and E3 regions are probably not involved in providing accessory functions (Carter et al., (1983) Virology 126:505). Adenoviruses defective in the E1 region, or having a deleted E4 region, are unable to support AAV replication. Thus, E1A and E4 regions appear necessary for AAV replication, either directly or indirectly (Laughlin et al., (1982) J. Virol. 41:868; Janik et al., (1981) Proc. Natl. Acad. Sci. USA 78:1925; Carter et al., (1983) Virology 126:505). Other characterized Adenovirus mutants include: E1B (Laughlin et al. (1982), supra; Janik et al. (1981), supra; Ostrove et al., (1980) Virology 104:502); E2A (Handa et al., (1975) J. Gen. Virol. 29:239; Strauss et al., (1976) J. Virol. 17:140; Myers et al., (1980) J. Virol. 35:665; Jay et al., (1981) Proc. Natl. Acad. Sci. USA 78:2927; Myers et al., (1981) J. Biol. Chem. 256:567); E2B (Carter, Adeno-Associated Virus Helper Functions, in I CRC Handbook of Parvoviruses (P. Tijssen ed., 1990)); E3 (Carter et al. (1983), supra); and E4 (Carter et al. (1983), supra; Carter (1995)). Studies of the accessory functions provided by adenoviruses having mutations in the E1B coding region have produced conflicting results, but E1B55k may be required for AAV virion production, while E1B19k is not (Samulski et al., (1988) J. Virol. 62:206-210). In addition, International Publication WO 97/17458 and Matshushita et al., (1998) Gene Therapy 5:938-945, describe accessory function vectors encoding various Adenovirus genes. Exemplary accessory function vectors comprise an adenovirus VA RNA coding region, an adenovirus E4 ORF6 coding region, an adenovirus E2A 72 kD coding region, an adenovirus E1A coding region, and an adenovirus E1B region lacking an intact E1B55k coding region. Such accessory function vectors are described, for example, in International Publication No. WO 01/83797.

As used herein, the term “serotype” is a distinction used to refer to an AAV having a capsid that is serologically distinct from other AAV serotypes. Serologic distinctiveness is determined on the basis of the lack of cross-reactivity between antibodies to one AAV as compared to another AAV. Cross-reactivity differences are usually due to differences in capsid protein sequences/antigenic determinants (e.g., due to VP1, VP2, and/or VP3 sequence differences of AAV serotypes).

Under the traditional definition, a serotype means that the virus of interest has been tested against serum specific for all existing and characterized serotypes for neutralizing activity and no antibodies have been found that neutralize the virus of interest. As more naturally occurring virus isolates of are discovered and/or capsid mutants generated, there may or may not be serological differences with any of the currently existing serotypes. Thus, in cases where the new virus (e.g., AAV) has no serological difference, this new virus (e.g., AAV) would be a subgroup or variant of the corresponding serotype. In many cases, serology testing for neutralizing activity has yet to be performed on mutant viruses with capsid sequence modifications to determine if they are of another serotype according to the traditional definition of serotype. Accordingly, for the sake of convenience and to avoid repetition, the term “serotype” broadly refers to both serologically distinct viruses (e.g., AAV) as well as viruses (e.g., AAV) that are not serologically distinct that may be within a subgroup or a variant of a given serotype.

rAAV vectors include any viral strain or serotype. As a non-limiting example, a rAAV vector genome or particle (capsid, such as VP1, VP2 and/or VP3) can be based upon any AAV serotype, such as AAV-1, -2, -3, -4, -5, -6, -7, -8, -9, -10, -11, -12, -rh74, -rh10 or AAV-2i8, for example. Such vectors can be based on the same of strain or serotype (or subgroup or variant), or be different from each other. As a non-limiting example, a rAAV plasmid or vector genome or particle (capsid) based upon one serotype genome can be identical to one or more of the capsid proteins that package the vector. In addition, a rAAV plasmid or vector genome can be based upon an AAV serotype genome distinct from one or more of the capsid proteins that package the vector genome, in which case at least one of the three capsid proteins could be a different AAV serotype, e.g., AAV1, AAV2, AAV3, AAV4, AAV5, AAV6, AAV7, AAV8, AAV9, AAV10, AAV11, AAV12, -rh74, -rh10 or AAV-2i8, or variant thereof, for example. More specifically, a rAAV2 vector genome can comprise AAV2 ITRs but capsids from a different serotype, such as AAV1, AAV3, AAV4, AAV5, AAV6, AAV7, AAV8, AAV9, AAV10, AAV11, AAV12, -rh74, -rh10 or AAV-2i8, or variant thereof, for example. Accordingly, rAAV vectors include gene/protein sequences identical to gene/protein sequences characteristic for a particular serotype, as well as mixed serotypes also referred to as pseudotypes.

In various exemplary embodiments, a rAAV vector includes or consists of a capsid sequence at least 70% or more (e.g., 75%, 80%, 85%, 90%, 95%, 96%, 97%, 98%, 99%, 99.5%, etc.) identical to one or more AAV1, AAV2, AAV3, AAV4, AAV5, AAV6, AAV7, AAV8, AAV9, AAV10, AAV11, AAV12, -rh74, -rh10 or AAV-2i8, capsid proteins (VP1, VP2, and/or VP3 sequences). In various exemplary embodiments, a rAAV vector includes or consists of a sequence at least 70% or more (e.g., 75%, 80%, 85%, 90%, 95%, 96%, 97%, 98%, 99%, 99.5%, etc.) identical to one or more AAV1, AAV2, AAV3, AAV4, AAV5, AAV6, AAV7, AAV8, AAV9, AAV10, AAV11, AAV12, -rh74, -rh10 or AAV-2i8, ITR(s).

In particular embodiments, rAAV vectors include AAV1, AAV2, AAV3, AAV4, AAV5, AAV6, AAV7, AAV8, AAV9, AAV10, AAV11, AAV12, Rh10, Rh74 and AAV-2i8 variants (e.g., ITR and capsid variants, such as amino acid insertions, additions, substitutions and deletions) thereof, for example, as set forth in WO 2013/158879 (International Application PCT/US2013/037170), WO 2015/013313 (International Application PCT/US2014/047670) and US 2013/0059732 (U.S. application Ser. No. 13/594,773, discloses LK01, LK02, LK03, etc.). rAAV, such as AAV1, AAV2, AAV3, AAV4, AAV5, AAV6, AAV7, AAV8, AAV9, AAV10, AAV11, AAV12, -rh74, -rh10 or AAV-2i8 and variants, hybrids and chimeric sequences, can be constructed using recombinant techniques that are known to the skilled artisan, to include one or more heterologous polynucleotide sequences (transgenes) flanked with one or more functional AAV ITR sequences. Such AAV vectors typically retain at least one functional flanking ITR sequence(s), as necessary for the rescue, replication, and packaging of the recombinant vector into a rAAV vector particle. A rAAV vector genome would therefore include sequences required in cis for replication and packaging (e.g., functional ITR sequences).

As used herein the phrase “bona fide AAV vector” or “bona fide rAAV vector” refers to AAV vectors comprising a heterologous nucleic acid which are capable of infecting target cells. The phrase excludes empty AAV vectors (no heterologous nucleic acid), and AAV vectors lacking full inserts (e.g., heterologous nucleic acid fragments) or those AAV vectors containing host cell nucleic acids.

The terms “nucleic acid” and “polynucleotide” are used interchangeably herein to refer to all forms of nucleic acid, oligonucleotides, including deoxyribonucleic acid (DNA) and ribonucleic acid (RNA). Nucleic acids include genomic DNA, cDNA and antisense DNA, and spliced or unspliced mRNA, rRNA tRNA and inhibitory DNA or RNA (RNAi, e.g., small or short hairpin (sh)RNA, microRNA (miRNA), small or short interfering (si)RNA, trans-splicing RNA, or antisense RNA). Nucleic acids include naturally occurring, synthetic, and intentionally modified or altered polynucleotides. Nucleic acids can be single, double, or triplex, linear or circular, and can be of any length. In discussing nucleic acids, a sequence or structure of a particular polynucleotide may be described herein according to the convention of providing the sequence in the 5′ to 3′ direction.

A “heterologous” nucleic acid sequence refers to a polynucleotide inserted into an AAV plasmid or vector for purposes of vector mediated transfer/delivery of the polynucleotide into a cell. Heterologous nucleic acid sequences are distinct from AAV nucleic acid, i.e., are non-native with respect to AAV nucleic acid. Once transferred/delivered into the cell, a heterologous nucleic acid sequence, contained within the vector, can be expressed (e.g., transcribed, and translated if appropriate). Alternatively, a transferred/delivered heterologous polynucleotide in a cell, contained within the vector, need not be expressed. Although the term “heterologous” is not always used herein in reference to nucleic acid sequences and polynucleotides, reference to a nucleic acid sequence or polynucleotide even in the absence of the modifier “heterologous” is intended to include heterologous nucleic acid sequences and polynucleotides in spite of the omission.

The “polypeptides,” “proteins” and “peptides” encoded by the “nucleic acid sequence” such as a heterologous nucleic acid sequence include full-length native sequences, as with naturally occurring proteins, as well as functional subsequences, modified forms or sequence variants so long as the subsequence, modified form or variant retains some degree of functionality of the native full-length protein. Such polypeptides, proteins and peptides encoded by the nucleic acid sequences can be but are not required to be identical to the endogenous protein that is defective, or whose expression is insufficient, or deficient in the treated mammal.

A “transgene” is used herein to conveniently refer to a nucleic acid (e.g., heterologous) that is intended or has been introduced into a cell or organism. Transgenes include any nucleic acid, such as a heterologous nucleic acid encoding a therapeutic protein or polypeptide sequence.

In a cell having a transgene, the transgene has been introduced/transferred by way of a plasmid or an AAV vector, “transduction” or “transfection” of the cell. The terms “transduce” and “transfect” refer to introduction of a molecule such as a nucleic acid into a host cell (e.g., HEK293) or cells or organ of an organism. The transgene may or may not be integrated into genomic nucleic acid of the recipient cell.

A “host cell” denotes, for example, microorganisms, yeast cells, insect cells, and mammalian cells, that can be, or have been, used as recipients of an AAV vector plasmid, AAV helper construct, an accessory function vector, or other transfer DNA. The term includes the progeny of the original cell which has been transfected. Thus, a “host cell” generally refers to a cell which has been transfected with an exogenous DNA sequence. It is understood that the progeny of a single parental cell may not necessarily be completely identical in morphology or in genomic or total DNA complement as the original parent, due to natural, accidental, or deliberate mutation. Exemplary host cells include human embryonic kidney (HEK) cells such as HEK293.

A “transduced cell” is a cell into which a transgene has been introduced. Accordingly, a “transduced” cell means a genetic change in a cell following incorporation of an exogenous molecule, for example, a nucleic acid (e.g., a transgene) into the cell. Thus, a “transduced” cell is a cell into which, or a progeny thereof in which an exogenous nucleic acid has been introduced. The cell(s) can be propagated (cultured) and the introduced protein expressed or nucleic acid transcribed, or vector, such as rAAV, produced by the cell. For gene therapy uses and methods, a transduced cell can comprise an organ or tissue and in turn can be in a subject.

As used herein, the term “stable” in reference to a cell, or “stably integrated” means that nucleic acid sequences, such as a selectable marker or heterologous nucleic acid sequence, or plasmid or vector has been inserted into a chromosome (e.g., by homologous recombination, non-homologous end joining, transfection, etc.) or is maintained in the recipient cell or host organism extrachromosomally, and has remained in the chromosome or is maintained extrachromosomally for a period of time.

A “cell line” refers to a population of cells capable of continuous or prolonged growth and division in vitro under appropriate culture conditions. Cell lines can, but need not be, clonal populations derived from a single progenitor cell. In cell lines, spontaneous or induced changes can occur in karyotype during storage or transfer of such clonal populations, as well as during prolonged passaging in tissue culture. Thus, progeny cells derived from the cell line may not be precisely identical to the ancestral cells or cultures. An exemplary cell line applicable to the invention purification methods is HEK293.

An “expression control element” refers to nucleic acid sequence(s) that influence expression of an operably linked nucleic acid. Control elements include expression control elements as set forth herein such as promoters and enhancers. rAAV vectors can include one or more “expression control elements.” Typically, such elements are included to facilitate proper heterologous polynucleotide transcription and, if appropriate, translation (e.g., one or more of a promoter, enhancer, splicing signal for introns, maintenance of the correct reading frame of the gene to permit in-frame translation of mRNA and stop codons, etc.). Such elements typically act in cis, referred to as a “cis acting” element, but may also act in trans.

Expression control can be effected at the level of transcription, translation, splicing, message stability, etc. Typically, an expression control element that modulates transcription is juxtaposed near the 5′ end (i.e., “upstream”) of a transcribed nucleic acid. Expression control elements can also be located at the 3′ end (i.e., “downstream”) of the transcribed sequence or within the transcript (e.g., in an intron). Expression control elements can be located adjacent to or at a distance away from the transcribed sequence (e.g., 1-10, 10-25, 25-50, 50-100, 100 to 500, or more nucleotides from the polynucleotide), even at considerable distances from the 5′ or 3′ end. Nevertheless, owing to the length limitations of rAAV vectors, expression control elements will typically be within 1 to 1000 nucleotides from the transcribed nucleic acid.

Functionally, expression of operably linked nucleic acid is at least in part controllable by the element (e.g., promoter, enhancer, etc.) such that the element modulates transcription of the nucleic acid and, as appropriate, translation of the transcript. A specific example of an expression control element is a promoter, which is usually located 5′ of the transcribed sequence. A promoter typically increases expression from operably linked nucleic acid as compared to an amount (if any) expressed when no promoter exists.

An “enhancer” as used herein can refer to a sequence that is located adjacent to the nucleic acid sequence, such as a heterologous nucleic acid sequence. Enhancer elements are typically located upstream (5′) of a promoter element but also function and can be located downstream (3′) of or within a sequence. Hence, an enhancer element can be located upstream or downstream, e.g., within 100 base pairs, 200 base pairs, or 300 or more base pairs of the as selectable marker, and/or a heterologous nucleic acid encoding a therapeutic protein or polypeptide sequence. Enhancer elements typically increase expression of an operably linked nucleic acid above expression afforded by a promoter element.

The term “operably linked” means that the regulatory sequences necessary for expression of a nucleic acid sequence are placed in the appropriate positions relative to the sequence so as to effect expression of the nucleic acid sequence. This same definition is sometimes applied to the arrangement of nucleic acid sequences and transcription control elements (e.g., promoters, enhancers, and termination elements) in an expression vector, e.g., rAAV vector.

In the example of an expression control element in operable linkage with a nucleic acid, the relationship is such that the control element modulates expression of the nucleic acid. More specifically, for example, two DNA sequences operably linked means that the two DNAs are arranged (cis or trans) in such a relationship that at least one of the DNA sequences is able to exert a modulatory effect upon the other sequence.

Accordingly, additional elements for vectors include, without limitation, an expression control (e.g., promoter/enhancer) element, a transcription termination signal or stop codon, 5′ or 3′ untranslated regions (e.g., polyadenylation (polyA) sequences) which flank a sequence (e.g., heterologous sequence), such as one or more copies of an AAV ITR sequence, or an intron.

Further elements include, for example, filler or stuffer polynucleotide sequences, for example to improve packaging and reduce the presence of contaminating nucleic acid. AAV vectors typically accept inserts of DNA having a size range which is generally about 4 kb to about 5.2 kb, or slightly more. Thus, for shorter sequences, a stuffer or filler is included in order to adjust the length to near or at the normal size of the virus genomic sequence acceptable for vector packaging into a rAAV particle. In various embodiments, a filler/stuffer nucleic acid sequence is an untranslated (non-protein encoding) segment of nucleic acid. For a nucleic acid sequence less than 4.7 Kb, the filler or stuffer polynucleotide sequence has a length that when combined (e.g., inserted into a vector) with the sequence has a total length between about 3.0-5.5 Kb, or between about 4.0-5.0 Kb, or between about 4.3-4.8 Kb.

A “therapeutic protein” in one embodiment is a peptide or protein that may alleviate or reduce symptoms that result from an insufficient amount, absence or defect in a protein in a cell or subject. A “therapeutic” protein encoded by a transgene can confer a benefit to a subject, e.g., to correct a genetic defect, to correct a gene (loss of expression or function) deficiency, etc.

Non-limiting examples of heterologous nucleic acids encoding gene products (e.g., therapeutic proteins) useful in accordance with the invention include those that may be used in the treatment of a disease or disorder including, but not limited to, “hemostasis” or blood clotting (bleeding) disorders such as hemophilia A, hemophilia A patients with inhibitory antibodies, hemophilia B, deficiencies in blood coagulation Factors VII, VIII, IX, X, XI, V, XII, II, von Willebrand factor, combined FV/FVIII deficiency, thalassemia, vitamin K epoxide reductase C1 deficiency, gamma-carboxylase deficiency; anemia, bleeding associated with trauma, injury, thrombosis, thrombocytopenia, stroke, coagulopathy, disseminated intravascular coagulation (DIC); over-anticoagulation associated with heparin, low molecular weight heparin, pentasaccharide, warfarin, small molecule antithrombotics (i.e., FXa inhibitors); and platelet disorders such as, Bernard Soulier syndrome, Glanzmann thrombasthenia, and storage pool deficiency.

Other non-limiting examples of heterologous nucleic acids encoding gene products (e.g., therapeutic proteins) useful in accordance with the invention include those which may optionally be expressed in liver or liver cells (e.g., hepatocytes) and provide a benefit include: GAA (acid alpha-glucosidase) for treatment of Pompe disease; ATP7B (copper transporting ATPase2) for treatment of Wilson's disease; alpha galactosidase for treatment of Fabry's disease; ASS1 (arginosuccinate synthase) for treatment of Citrullinemia Type 1; beta-glucocerebrosidase for treatment of Gaucher disease Type 1; beta-hexosaminidase A for treatment of Tay Sachs disease; SERPING1 (C1 protease inhibitor) for treatment of Hereditary Angioedema; glucose-6-phosphatase for treatment of glycogen storage disease type I (GSDI); erythropoietin (EPO) for treatment of anemia; interferon-alpha, interferon-beta, and interferon-gamma for treatment of various immune disorders, viral infections and cancer; an interleukin (IL), including any one of IL-1 through IL-36, and corresponding receptors, for treatment of various inflammatory diseases or immuno-deficiencies; a chemokine, including chemokine (C-X-C motif) ligand 5 (CXCL5), for treatment of immune disorders; granulocyte-colony stimulating factor (G-CSF) for treatment of immune disorders such as Crohn's disease; granulocyte-macrophage colony stimulating factor (GM-CSF) for treatment of various human inflammatory diseases; macrophage colony stimulating factor (M-CSF) for treatment of various human inflammatory diseases; keratinocyte growth factor (KGF) for treatment of epithelial tissue damage; chemokines such as monocyte chemoattractant protein-1 (MCP-1) for treatment of recurrent miscarriage, HIV-related complications, and insulin resistance, for example; tumor necrosis factor (TNF) and receptors for treatment of various immune disorders; alpha-1 antitrypsin for treatment of emphysema or chronic obstructive pulmonary disease (COPD); alpha-L-iduronidase for treatment of Mucopolysaccharidosis I (MPS I); ornithine transcarbamoylase for treatment of OTC deficiency; phenylalanine hydroxylase (PAH) or phenylalanine ammonia-lyase (PAL) for treatment of Phenylketonuria (PKU); lipoprotein lipase for treatment of lipoprotein lipase deficiency; apolipoproteins for treatment of apolipoprotein (Apo) A-I deficiency; low-density lipoprotein receptor (LDL-R) for treatment of Familial hypercholesterolemia (FH); albumin for treatment of Hypoalbuminemia; lecithin cholesterol acyltransferase (LCAT); carbamoyl synthetase I; argininosuccinate synthetase; argininosuccinate lyase; arginase; fumarylacetoacetate hydrolase; porphobilinogen deaminase; cystathionine beta-synthase, for treatment of homocystinuria; branched chain ketoacid decarboxylase; isovaleryl-CoA dehydrogenase; propionyl CoA carboxylase; methylmalonyl-CoA mutase; glutaryl CoA dehydrogenase; insulin; pyruvate carboxylase; hepatic phosphorylase; phosphorylase kinase; glycine decarboxylase; H-protein, T-protein, cystic fibrosis transmembrane regulator (CFTR); dystrophin, microdystrophin or CT GaINAc transferase (Galgt2) for the treatment of Duchenne muscular dystrophy; and survival motor neuron (SMN) for the treatment of spinal muscular atrophy (SMA).

Further non-limiting examples of heterologous nucleic acids encoding gene products (e.g., therapeutic proteins) useful in accordance with the invention and relating to ocular diseases or conditions include: rhodopsin, phosphodiesterase 613, ATP-binding cassette, sub-family A, member 4, retinal pigment epithelium-specific 65 kDa protein, lecithin retinal acyltransferase, retinal degeneration, slow/peripherin, tyrosine-protein kinase MER, cGMP-gated cation channel alpha-1, retinitis pigmentosa GTPase regulator, inosine-5-prime-monophosphate dehydrogenase, type I, or channelrhodopsin-2 for treatment of retinitis pigmentosa; guanylate cyclase 2D, retinal degeneration, slow/peripherin, aryl-hydrocarbon interacting protein-like 1, or ATP-binding cassette, sub-family A, member 4 for treatment of maculopathies; inosine-5-prime-monophosphate dehydrogenase, type I, aryl-hydrocarbon interacting protein-like 1, guanylate cyclase 2D, lecithin retinal acyltransferase, tyrosine-protein kinase MER, retinitis pigmentosa GTPase regulator interacting protein 1, retinal pigment epithelium-specific 65 kDa protein, or centrosomal protein of 290 kDa for treatment of Leber's congenital amaurosis and early onset severe retinal dystrophy; ATP-binding cassette, sub-family A, member 4 (ABCA4) for treatment of Stargardt disease; whirlin, myosin 7A, harmonin, cadherin 23, protocadherin 15, Usher syndrome type-1G Protein (SANS), or clarin 1 for treatment of Usher syndrome; G protein subunit alpha transducin 2 (GNAT2), cyclic nucleotide gated channel alpha 3 (CNGA3), or cyclic nucleotide gated channel beta 3 (CNGB3) for treatment of achromatopsia; retinoschisin 1 (RS1) for treatment of X-linked retinoschisis; ocular albinism type 1 (OA1) protein for treatment of ocular albinism (also known as Nettleship-Falls syndrome); NADH-ubiquinone oxidoreductase chain 4 (MT-ND4) for treatment of Leber's hereditary optic neuropathy; oculocutaneous albinism type 1 (OCA1) tyrosinase for treatment of oculocutaneous albinism; cyclin-dependent kinase inhibitor interacting protein 1 (p21 WAF-1) for treatment of glaucoma; Rab escort protein 1 (REP-1) for treatment of choroideremia; platelet-derived growth factor, endostatin, angiostatin or vascular endothelial growth factor inhibitor for treatment of age related macular degeneration; opsin for treatment of color blindness; long-wave-sensitive opsin 1 for treatment of blue cone monochromacy; arylsulfatase B for treatment of lysosomal storage disease IV; and β-glucuronidase for treatment of lysosomal storage disease VII.

Other non-limiting examples of heterologous nucleic acids encoding gene products useful in accordance with the invention include reporters or detectable markers such as luciferase, green fluorescent protein (GFP), yellow fluorescent protein (YFP), blue fluorescent protein, cyan fluorescent protein, enhanced GFP, enhanced YFP, photoactivatable GFP, Discosoma species fluorescent protein (dsRed), mFruits, mCherry, TagRFPs, eqFP611, photoswitchable fluorescent proteins (for example Dronpa and EosFP), chloramphenicol acetyltransferase, Halo-tag fusion protein, alkaline phosphatase, horseradish peroxidase and beta-galactosidase.

Nucleic acid molecules, vectors such as cloning, expression vectors (e.g., vector genomes) and plasmids, may be prepared using recombinant DNA technology methods. The availability of nucleotide sequence information enables preparation of nucleic acid molecules by a variety of means. For example, a heterologous nucleic acid comprising a vector or plasmid can be made using various standard cloning, recombinant DNA technology, via cell expression or in vitro translation and chemical synthesis techniques. Purity of polynucleotides can be determined through sequencing, gel electrophoresis and the like. For example, nucleic acids can be isolated using hybridization or computer-based database screening techniques. Such techniques include, but are not limited to: (1) hybridization of genomic DNA or cDNA libraries with probes to detect homologous nucleotide sequences; (2) antibody screening to detect polypeptides having shared structural features, for example, using an expression library; (3) polymerase chain reaction (PCR) on genomic DNA or cDNA using primers capable of annealing to a nucleic acid sequence of interest; (4) computer searches of sequence databases for related sequences; and (5) differential screening of a subtracted nucleic acid library.

The term “isolated,” when used as a modifier of a composition, means that the compositions are made by the hand of man or are separated, completely or at least in part, from their naturally occurring in vivo environment. Generally, isolated compositions are substantially free of one or more materials with which they normally associate with in nature, for example, one or more protein, nucleic acid, lipid, carbohydrate, cell membrane.

With respect to protein, the term “isolated protein” or “isolated and purified protein” is sometimes used herein. This term refers primarily to a protein produced by expression of a nucleic acid molecule. Alternatively, this term may refer to a protein which has been sufficiently separated from other proteins with which it would naturally be associated, so as to exist in “substantially pure” form.

The term “isolated” does not exclude compositions herein or combinations produced by the hand of man, for example, a rAAV, LNP and/or a pharmaceutical formulation. The term “isolated” also does not exclude alternative physical forms of the composition, such as hybrids/chimeras, multimers/oligomers, modifications (e.g., phosphorylation, glycosylation, lipidation) or derivatized forms, or forms expressed in host cells produced by the hand of man.

The term “substantially pure” refers to a preparation comprising at least 50-60% by weight the compound of interest (e.g., nucleic acid, oligonucleotide, protein, etc.). The preparation can comprise at least 75% by weight, or about 90-99% by weight, of the compound of interest. Purity is measured by methods appropriate for the compound of interest (e.g., chromatographic methods, agarose or polyacrylamide gel electrophoresis, HPLC analysis, and the like).

The phrase “consisting essentially of” when referring to a particular nucleotide sequence or amino acid sequence means a sequence having the properties of a given sequence. For example, when used in reference to a nucleic acid or an amino acid sequence, the phrase includes the sequence per se and molecular modifications that would not affect the basic and novel characteristics of the sequence.

Methods that are known in the art for generating rAAV virions include, for example, transfection using AAV vector and AAV helper sequences in conjunction with coinfection with one or more AAV helper virus(es) (e.g., adenovirus, herpesvirus, or vaccinia virus) or transfection with a recombinant AAV vector, an AAV helper vector, and an accessory function vector. Non-limiting methods for generating rAAV virions are described, for example, in U.S. Pat. Nos. 6,001,650 and 6,004,797. Following rAAV vector production (i.e., vector generation in cell culture systems), rAAV virions can be obtained from the host cells and cell culture supernatant and purified as according to methods known in the art.

Methods to determine infectious titer of rAAV vector containing a transgene are known in the art (See, e.g., Zhen et al., (2004) Hum. Gene Ther. (2004) 15:709). Methods for assaying for empty capsids and AAV vector particles with packaged genomes are known (See, e.g., Grimm et al., Gene Therapy (1999) 6:1322-1330; Sommer et al., Molec. Ther. (2003) 7:122-128).

To determine degraded/denatured capsid, purified rAAV can be subjected to SDS-polyacrylamide gel electrophoresis, consisting of any gel capable of separating the three capsid proteins, for example, a gradient gel, then running the gel until sample is separated, and blotting the gel onto nylon or nitrocellulose membranes. Anti-AAV capsid antibodies are then used as primary antibodies that bind to denatured capsid proteins (See, e.g., Wobus et al., J. Virol. (2000) 74:9281-9293). A secondary antibody that binds to the primary antibody contains a means for detecting the primary antibody. Binding between the primary and secondary antibodies is detected semi-quantitatively to determine the amount of capsids.

The pI (isoelectric point) of AAV is in a range from about 6 to about 6.5. Thus, the AAV surface carries a slight negative charge. As such it may be beneficial for the LNP to comprise a cationic lipid. In certain embodiments, the cationic lipid comprises an amino lipid. Exemplary amino lipids have been described in U.S. Pat. Nos. 9,352,042, 9,220,683, 9,186,325, 9,139,554, 9,126,966 9,018,187, 8,999,351, 8,722,082, 8,642,076, 8,569,256, 8,466,122, and 7,745,651 and U.S. Patent Publication Nos. 2016/0213785, 2016/0199485, 2015/0265708, 2014/0288146, 2013/0123338, 2013/0116307, 2013/0064894, 2012/0172411, and 2010/0117125, all of which are incorporated herein by reference in their entirety.

The terms “cationic lipid” and “amino lipid” are used interchangeably herein to include those lipids and salts thereof having one, two, three, or more fatty acid or fatty alkyl chains and a pH-titratable amino group (e.g., an alkylamino or dialkylamino group). The cationic lipid is typically protonated (i.e., positively charged) at a pH below the pKₐ of the cationic lipid and is substantially neutral at a pH above the pKₐ. The cationic lipids may also be titratable cationic lipids. In some embodiments, the cationic lipids comprise: a protonatable tertiary amine (e.g., pH-titratable) group; 018 alkyl chains, wherein each alkyl chain independently has 0 to 3 (e.g., 0, 1, 2, or 3) double bonds; and ether, ester, or ketal linkages between the head group and alkyl chains.

Cationic lipids may include, without limitation, 1,2-dilinoleyloxy-N,N-dimethylaminopropane (DLinDMA), 1,2-dilinolenyloxy-N,N-dimethylaminopropane (DLenDMA), 1,2-di-γ-linolenyloxy-N,N-dimethylaminopropane (γ-DLenDMA), 2,2-dilinoleyl-4-(2-dimethylaminoethyl)-[1,3]-dioxolane (DLin-K-C2-DMA, also known as DLin-C2K-DMA, XTC2, and C2K), 2,2-dilinoleyl-4-dimethylaminomethyl-[1,3]-dioxolane (DLin-K-DMA), dilinoleylmethyl-3-dimethylaminopropionate (DLin-M-C2-DMA, also known as MC2), (6Z,9Z,28Z,31 Z)-heptatriaconta-6,9,28,31-tetraen-19-yl 4-(dimethylamino)butanoate (DLin-M-C3-DMA, also known as MC3), salts thereof, and mixtures thereof. Other cationic lipids also include, but are not limited to, 1,2-distearyloxy-N,N-dimethyl-3-aminopropane (DSDMA), 1,2-dioleyloxy-N,N-dimethyl-3-aminopropane (DODMA), 2,2-dilinoleyl-4-(3-dimethylaminopropyl)-[1,3]-dioxolane (DLin-K-C3-DMA), 2,2-dilinoleyl-4-(3-dimethylaminobutyl)-[1,3]-dioxolane (DLin-K-C4-DMA), DLen-C2K-DMA, γ-DLen-C2K-DMA, and (DLin-MP-DMA) (also known as 1-611).

Still further cationic lipids may include, without limitation, 2,2-dilinoleyl-5-dimethylaminomethyl-[1,3]-dioxane (DLin-K6-DMA), 2,2-dilinoleyl-4-N-methylpepiazino-[1,3]-dioxolane (DLin-K-MPZ), 1,2-dilinoleylcarbamoyloxy-3-dimethylaminopropane (DLin-C-DAP), 1,2-dilinoleyoxy-3-(dimethylamino)acetoxypropane (DLin-DAC), 1,2-dilinoleyoxy-3-morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3-dimethylaminopropane (DLin-2-DMAP), 1,2-dilinoleyloxy-3-trimethylaminopropane chloride salt (DLin-TMA.Cl), 1,2-dilinoleoyl-3-trimethylaminopropane chloride salt (DLin-TAP.Cl), 1,2-dilinoleyloxy-3-(N-methylpiperazino)propane (DLin-MPZ), 3-(N,N-dilinoleylamino)-1,2-propanediol (DLinAP), 3-(N,N-dioleylamino)-1,2-propanedio (DOAP), 1,2-dilinoleyloxo-3-(2-N,N-dimethylamino)ethoxypropane (DLin-EG-DMA), N,N-dioleyl-N,N-dimethylammonium chloride (DODAC), N-(1-(2,3-dioleyloxy)propyl)-N,N,N-trimethylammonium chloride (DOTMA), N,N-distearyl-N,N-dimethylammonium bromide (DDAB), N-(1-(2,3-dioleoyloxy)propyl)-N,N,N-trimethylammonium chloride (DOTAP), 3-(N—(N′,N′-dimethylaminoethane)-carbamoyl)cholesterol (DC-Chol), N-(1,2-dimyristyloxyprop-3-yl)-N,N-dimethyl-N-hydroxyethyl ammonium bromide (DMRIE), 2,3-dioleyloxy-N-[2(spermine-carboxamido)ethyl]-N,N-dimethyl-1-propanaminiumtrifluoroacetate (DOSPA), dioctadecylamidoglycyl spermine (DOGS), 3-dimethylamino-2-(cholest-5-en-3-beta-oxybutan-4-oxy)-1-(cis,cis-9,12-octadecadienoxy)propane (CLinDMA), 2-[5′-(cholest-5-en-3-beta-oxy)-3′-oxapentoxy)-3-dimethyl-1-(cis,cis-9′,1-2′-octadecadienoxy)propane (CpLinDMA), N,N-dimethyl-3,4-dioleyloxybenzylamine (DMOBA), 1,2-N,N′-dioleylcarbamyl-3-dimethylaminopropane (DOcarbDAP), 1,2-N,N′-dilinoleylcarbamyl-3-dimethylaminopropane (DLincarbDAP), dexamethasone-spermine (DS) and disubstituted spermine (D2S) or mixtures thereof.

Additionally, a number of commercial preparations of cationic lipids can be used, such as, LIPOFECTIN® (including DOTMA and DOPE, available from GIBCO/BRL), and LIPOFECTAMINE® (comprising DOSPA and DOPE, available from GIBCO/BRL).

In certain embodiments, the cationic lipid may be present in an amount from about 10% by weight of the LNP to about 85% by weight of the lipid nanoparticle, or from about 50% by weight of the LNP to about 75% by weight of the LNP.

In various embodiments, the LNP comprises a sterol. Sterols may confer fluidity to the LNP. As used herein, “sterol” refers to any naturally occurring sterol of plant (phytosterols) or animal (zoosterols) origin as well as non-naturally occurring synthetic sterols, all of which are characterized by the presence of a hydroxyl group at the 3-position of the steroid A-ring. The sterol can be any sterol conventionally used in the field of liposome, lipid vesicle or lipid particle preparation, which is most commonly cholesterol. Phytosterols may include campesterol, sitosterol, and stigmasterol. Sterols also includes sterol-modified lipids, such as those described in U.S. Patent Application Publication 2011/0177156, which is incorporated herein by reference in its entirety. In some embodiments, a sterol may be present in an amount from about 5% by weight of the LNP to about 50% by weight of the lipid nanoparticle or from about 10% by weight of the LNP to about 25% by weight of the LNP.

In some embodiments, the LNP comprises a neutral lipid. Neutral lipids may comprise any lipid species which exists either in an uncharged or neutral zwitterionic form at physiological pH. Such lipids include, without limitation, diacylphosphatidylcholine, diacylphosphatidylethanolamine, ceramide, sphingomyelin, dihydrosphingomyelin, cephalin, and cerebrosides. The selection of neutral lipids for use in the lipid nanoparticles described herein is generally guided by consideration of, inter alia, particle size and the requisite stability. In some embodiments, the neutral lipid component may be a lipid having two acyl groups, (e.g., diacylphosphatidylcholine and diacylphosphatidylethanolamine). Lipids having a variety of acyl chain groups of varying chain length and degree of saturation are available or may be isolated or synthesized by well-known techniques. In some embodiments, lipids containing saturated fatty acids with carbon chain lengths in the range of C₁₄ to C₂₂ may be used. In another group of embodiments, lipids with mono or diunsaturated fatty acids with carbon chain lengths in the range of C₁₄ to C₂₂ are used. Additionally, lipids having mixtures of saturated and unsaturated fatty acid chains can be used. Exemplary neutral lipids include, without limitation, 1,2-dioleoyl-sn-glycero-3-phosphatidyl-ethanolamine (DOPE), 1,2-distearoyl-sn-glycero-3-phosphocholine (DSPC), 1-palmitoyl-2-oleoyl-sn-glycero-3-phosphocholine (POPC), or any related phosphatidylcholine. The neutral lipids useful in the invention may also be composed of sphingomyelin, dihydrosphingomyelin, or phospholipids with other head groups, such as serine and inositol.

In some embodiments, the neutral lipid may be present in an amount from about 0.1% by weight of the lipid nanoparticle to about 75% by weight of the LNP, or from about 5% by weight of the LNP to about 15% by weight of the LNP.

In some embodiments, the LNP may comprise a polyethylene glycol (PEG) or a PEG-modified lipid. The term “PEG” refers to a polyethylene glycol, a linear, water-soluble polymer of ethylene PEG repeating units with two terminal hydroxyl groups. PEGs are classified by their molecular weights; for example, PEG 2000 has an average molecular weight of about 2,000 daltons, and PEG 5000 has an average molecular weight of about 5,000 daltons. PEGs are commercially available from Sigma Chemical Co. and other companies and include, for example, the following functional PEGs: monomethoxypolyethylene glycol (MePEG-OH), monomethoxypolyethylene glycol-succinate (MePEG-S), monomethoxypolyethylene glycol-succinimidyl succinate (MePEG-S-NHS), monomethoxypolyethylene glycol-amine (MePEG-NH2), monomethoxypolyethylene glycol-tresylate (MePEG-TRES), and monomethoxypolyethylene glycol-imidazolyl-carbonyl (MePEG-IM).

In some embodiments, PEG may be a polyethylene glycol with an average molecular weight of about 550 to about 10,000 daltons and is optionally substituted by alkyl, alkoxy, acyl or aryl. In some embodiments, the PEG may be substituted with methyl at the terminal hydroxyl position. In another preferred embodiment, the PEG may have an average molecular weight from about 750 to about 5,000 daltons, or from about 1,000 to about 5,000 daltons, or from about 1,500 to about 3,000 daltons or from about 2,000 daltons or of about 750 daltons. The PEG can be optionally substituted with alkyl, alkoxy, acyl or aryl. In some embodiments, the terminal hydroxyl group may be substituted with a methoxy or methyl group.

PEG-modified lipids include the PEG-dialkyloxypropyl conjugates (PEG-DAA) described in U.S. Pat. Nos. 8,936,942 and 7,803,397, which are incorporated herein by reference in their entirety. PEG-modified lipids (or lipid-polyoxyethylene conjugates) that are useful may have a variety of “anchoring” lipid portions to secure the PEG portion to the surface of the lipid vesicle. Examples of suitable PEG-modified lipids include PEG-modified phosphatidylethanolamine and phosphatidic acid, PEG-ceramide conjugates (e.g., PEG-CerC₁₄ or PEG-CerC₂₀) which are described in U.S. Pat. No. 5,820,873, which is incorporated herein by reference, PEG-modified dialkylamines and PEG-modified 1,2-diacyloxypropan-3-amines. In some embodiments, the PEG-modified lipid may be PEG-modified diacylglycerols and dialkylglycerols. In some embodiments, the PEG may be in an amount from about 0.5% by weight of the LNP to about 20% by weight of the LNP, or from about 5% by weight of the LNP to about 15% by weight of the LNP.

In some embodiments, prior to encapsulating AAV the LNPs may have a size in a range from about 10 nm to 500 nm, or from about 50 nm to about 200 nm, or from 75 nm to about 125 nm. In some embodiments, LNP encapsulated AAV may have a size in a range from about 10 nm to 500 nm.

In some embodiments, LNP encapsulated AAV compositions may further comprise receptor-targeting functionality. In some such embodiments, the compositions incorporate a receptor-targeting moiety. Such targeting moieties which can bind a receptor may be covalently or non-covalently associated with the LNP. Selective receptors may be those disclosed in WO2015/081096, which is incorporated herein by reference in its entirety. Such exemplary selective receptors for targeting selective transport through tissue of the LNPs include, without limitation, gp60 for delivery to heart, skeletal muscle, or adipose tissue, chorionic gonadotropin receptor, insulin receptor and insulin-like growth, transferrin receptor for delivery to the testis, factor receptor, LDL receptor-related proteins 1 and 2 (LRP1 and LRP2), LDL receptor, diphtheria toxin receptor, transferrin, receptor for advanced glycation end products (RAGE), scavenger receptor (SR), receptors for M cells, terminal galactose (ricin B receptor), aminopeptidase N, plgA receptor, or cubulin/megalin (vitamin B12) for delivery to intestine, CD23 (for IgE) for delivery to the liver, plgA and terminal galactose (ricin B receptor), plgA, and gp60 for deliver to lung tissue, gp60, aminopeptidase N, and CD23 (for IgE) for delivery to mammary glands, gp60 for delivery to thyroid, and plgA, transferrin, megalin, gp340 and lutropin receptor for delivery to the genitourinary tract tissue.

LNP encapsulated AAV vector compositions disclosed herein may be prepared by numerous methods. In some embodiments, methods of making a composition comprising an AAV vector in a LNP comprise mixing an AAV vector with a LNP. In some such embodiments, the LNPs are preformed and then mixed with the AAV vector.

In some embodiments, the LNP is formed in the presence of AAV vector and the AAV vector is effectively encapsulated during LNP formation. In some such embodiments, methods of making a composition comprising an AAV vector in a LNP, comprises coating a vessel with lipid components and adding the AAV vector to the coated vessel. The LNP is then allowed to form around the AAV vector. In an exemplary embodiment, a method comprises solubilizing the lipid components of the LNP in an organic solvent such as ethanol, methanol, or chloroform. The lipid solution may be added to a vessel such as a test tube. Next, the organic solvent can be removed by heating, vacuum, or passing dry air or gas into the tube (or a combination of these methods) while rotating the vessel. The lipid components dry in a thin film on the sides of the vessel. AAV vector, in aqueous solution, may then be added to the vessel. The vessel is agitated by a method such as vortexing, sonication, etc. forming LNPs. During the formation of LNPs, AAV vector is encapsulated in the LNPs. LNPs can be made a homogeneous, defined size by extrusion through membranes.

LNP encapsulated AAV vector compositions disclosed herein may be prepared by lipid thin-film hydration or spray drying, followed by sonication, homogenization, including high pressure homogenization, or extrusion, in order to reduce particle size. LNP encapsulated AAV vector compositions may also be prepared by mixing an organic phase containing lipid components with an aqueous phase containing AAV vector to create an emulsion, followed by removal of the organic solvent to form the LNP encapsulated AAV. The organic solvent may be removed by evaporation, dialysis, filtration, or other methods known in the art. In other embodiments, lipid components are dissolved in an organic solvent, such as ethanol, and the organic solution is rapidly injected into an aqueous solution of AAV vector, optionally performed under varying heated temperature, to form LNP encapsulated AAV vector. Other procedures include double emulsion, freeze-thaw, dehydration-rehydration, fast extrusion and supercritical carbon dioxide, all of which are known in the art, such as that described in Kraft et al., J. Pharm. Sci. 103:29-52 (2014).

A vessel includes, without limitation, a tube, flask, vial, bottle, single or multiwall plate, dish, ampule.

Suitable vessel materials include, without limitation, glass and plastic. Suitable materials also include, without limitation, polyethylene, polybutylene, polystyrene, polycarbonate, polypropylene, polyester, silicone, etc.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

All applications, publications, patents and other references, GenBank citations and ATCC citations cited herein are incorporated by reference in their entirety. In case of conflict, the specification, including definitions, will control.

All of the features disclosed herein may be combined in any combination. Each feature disclosed in the specification may be replaced by an alternative feature serving a same, equivalent, or similar purpose. Thus, unless expressly stated otherwise, disclosed features (e.g., nucleic acid sequences, vectors, rAAV vectors, etc.) are an example of a genus of equivalent or similar features.

As used herein, the singular forms “a”, “and,” and “the” include plural referents unless the context clearly indicates otherwise. Thus, for example, reference to “an AAV vector,” or “AAV particle,” includes a plurality of such AAV vectors and AAV particles, and reference to “a nano-particle” or “lipid” includes a plurality of nano-particles and lipids.

The term “about” as used herein means values that are within 1-10% (plus or minus) of a reference value.

As used herein, all numerical values or numerical ranges include integers within such ranges and fractions of the values or the integers within ranges unless the context clearly indicates otherwise. Thus, to illustrate, reference to 80% or more identity, includes 81%, 82%, 83%, 84%, 85%, 86%, 87%, 88%, 89%, 90%, 91%, 92%, 93%, 94%, etc., as well as 81.1%, 81.2%, 81.3%, 81.4%, 81.5%, etc., 82.1%, 82.2%, 82.3%, 82.4%, 82.5%, etc., and so forth.

Reference to an integer with greater (more) or less (fewer) than includes any number greater or less than the reference number, respectively. Thus, for example, a reference to less than 100, includes 99, 98, 97, etc. all the way down to the number one (1); and less than 10, includes 9, 8, 7, etc. all the way down to the number one (1).

As used herein, all numerical values or ranges are inclusive. Further, all numerical values or ranges include fractions of the values and integers within such ranges and fractions of the integers within such ranges unless the context clearly indicates otherwise. Thus, to illustrate, reference to a numerical range, such as 1-10 includes 1, 2, 3, 4, 5, 6, 7, 8, 9, 10, as well as 1.1, 1.2, 1.3, 1.4, 1.5, etc., and so forth. Reference to a range of 1-50 therefore includes 1, 2, 3, 4, 5, 6, 7, 8, 9, 10, 11, 12, 13, 14, 15, 16, 17, 18, 19, 20, etc., up to and including 50, as well as 1.1, 1.2, 1.3, 1.4, 1.5, etc., 2.1, 2.2, 2.3, 2.4, 2.5, etc., and so forth.

Reference to a series of ranges includes ranges which combine the values of the boundaries of different ranges within the series. Thus, to illustrate reference to a series of ranges, for example, of 1-10, 10-20, 20-30, 30-40, 40-50, 50-60, 60-75, 75-100, 100-150, 150-200, 200-250, 250-300, 300-400, 400-500, 500-750, 750-1,000, 1,000-1,500, 1,500-2,000, 2,000-2,500, 2,500-3,000, 3,000-3,500, 3,500-4,000, 4,000-4,500, 4,500-5,000, 5,500-6,000, 6,000-7,000, 7,000-8,000, or 8,000-9,000, includes ranges of 10-50, 50-100, 100-1,000, 1,000-3,000, 2,000-4,000, etc.

The invention is generally disclosed herein using affirmative language to describe the numerous embodiments and aspects. The invention also specifically includes embodiments in which particular subject matter is excluded, in full or in part, such as substances or materials, method steps and conditions, protocols, or procedures. For example, in various embodiments or aspects of the invention, compositions, materials, methods and/or method steps are omitted or excluded. Thus, even though the invention is generally not expressed herein in terms of what the invention does not include, embodiments and aspects that are not expressly excluded in the invention are nevertheless disclosed herein.

A number of embodiments of the invention have been described. Nevertheless, one skilled in the art, without departing from the spirit and scope of the invention, can make various changes and modifications of the invention to adapt it to various usages and conditions. Accordingly, the following examples are intended to be illustrative only and are not intended to limit the scope of the present disclosure. Also, parts and percentages are by weight unless otherwise indicated. As used herein, “room temperature” refers to a temperature of from about 20° C. to about 25° C.

### EXAMPLES

### Example 1

This example describes a method of making LNP encapsulated AAV vector. In particular, a dry film hydrate method is described. In this method the lipid components of the LNP are solubilized in an organic solvent such as ethanol, methanol, or chloroform. The lipid solution is added to a vessel such as a test tube. The organic solvent is then removed by heating, vacuum, or passing dry air or gas into the tube (or a combination of these methods) while rotating the vessel. The lipid components dry in a thin film on the sides of the vessel. AAV vector, in aqueous solution, is then added to the vessel. The vessel is agitated by a method such as vortexing, sonication, etc. forming LNPs. During the formation of LNPs, AAV vector is encapsulated in the LNPs. LNPs can be made a homogeneous, defined size by extrusion through membranes.

### Example 2

This example describes a method of making Lipofectamine® encapsulated AAV vector. The encapsulation method can be performed according to the manufacturer's protocol (tools.thermofisher.com/content/sfs/manuals/Lipofectamine_2000_Reagprotocol.pdf). Briefly, an aqueous solution of Lipofectamineand AAV vector is incubated at room temperature for at least 5 minutes and LNP encapsulated AAV vector is formed.

### Example 3

Lipids are solubilized in an organic solvent, such as ethanol. AAV vector is solubilized in aqueous buffer. The organic solvent containing lipids and aqueous buffer containing AAV are flowed into a “T” junction from opposite sides. The resulting turbulent mixing produces LNPs with encapsulation of AAV vector which exit the bottom of the junction. Size of LNPs can be controlled by factors such as rate of flow and size of the junction.

### Example 4

Lipids are solubilized in an aqueous buffer by use of a detergent. AAV vector is added to the lipid/detergent solution. Detergent is removed by dialysis forming LNPs with encapsulated AAV.

### Example 5

Lipids are solubilized in ethanol and added dropwise to AAV in aqueous buffer while mixing to produce LNPs with encapsulated AAV vector.

What is claimed is:

1. A composition comprising an adeno-associated virus (AAV) vector in a lipid nanoparticle (LNP), said AAV vector comprising a heterologous nucleic acid sequence and an inverted terminal repeat (ITR) positioned 5′ of said heterologous nucleic acid sequence and an ITR positioned 3′ of said heterologous nucleic acid sequence.

2. The composition of claim 1, wherein said LNP comprises a cationic lipid.

3. The composition of claim 2, wherein said cationic lipid comprises an amino lipid.

4. The composition of claim 2, wherein said cationic lipid is present in an amount from about 10% by weight of said LNP to about 75% by weight of said LNP.

5. The composition of any of claims 1-4, wherein said LNP further comprises a sterol.

6. The composition of claim 5, wherein said sterol is present in an amount from about 5% by weight of said LNP to about 50% by weight of said LNP.

7. The composition of any of claims 1-6, wherein said LNP comprises a neutral lipid.

8. The composition of claim 7, wherein said neutral lipid is present in an amount from about 0.1% by weight of said LNP to about 15% by weight of said LNP.

9. The composition of any of claims 1-8, wherein said LNP comprises polyethylene glycol (PEG) or a PEG-modified lipid.

10. The composition of claim 9, wherein said PEG or PEG-modified lipid is present in an amount from about 0.5% by weight of said LNP to about 10% by weight of said LNP.

11. The composition of any of claims 1-10, wherein said LNP has a size in a range from about 10 nm to 500 nm.

12. The composition of any of claims 1-11, wherein said LNP has a size in a range from about 50 nm to 200 nm.

13. The composition of any of claims 1-12, wherein said LNP has a size in of about 100 nm.

14. The composition of any of claims 1-13, wherein said LNP further comprises a cell targeting or cell penetrating moiety.

15. The composition of any of claims 1-14, wherein said heterologous nucleic acid sequence comprises or encodes a blood coagulation Factor.

16. The composition of any of claims 1-14, wherein said heterologous nucleic acid sequence comprises or encodes a Factor VII, VIII, IX, X, XI, V, XII, II, von Willebrand factor, vitamin K epoxide reductase C1, or gamma-carboxylase.

17. The composition of any of claims 1-14, wherein said heterologous nucleic acid sequence comprises or encodes GAA (acid alpha-glucosidase); ATP7B (copper transporting ATPase2); alpha galactosidase; ASS1 (arginosuccinate synthase); beta-glucocerebrosidase; beta-hexosaminidase A; SERPING1 (C1 protease inhibitor); glucose-6-phosphatase; erythropoietin (EPO; interferon-alpha; interferon-beta; interferon-gamma; an interleukin (IL); any one of Interleukins 1-36 (IL-1 through IL-36); interleukin (IL) receptor; a chemokine; chemokine (C—X-C motif) ligand 5 (CXCL5); granulocyte-colony stimulating factor (G-CSF); granulocyte-macrophage colony stimulating factor (GM-CSF); macrophage colony stimulating factor (M-CSF); keratinocyte growth factor (KGF); monocyte chemoattractant protein-1 (MCP-1); tumor necrosis factor (TNF); a tumor necrosis factor (TNF) receptor; alpha-1 antitrypsin; alpha-L-iduronidase; ornithine transcarbamoylase; phenylalanine hydroxylase (PAH); phenylalanine ammonia-lyase (PAL); lipoprotein lipase; an apolipoprotein; low-density lipoprotein receptor (LDL-R); albumin; lecithin cholesterol acyltransferase (LCAT); carbamoyl synthetase I; argininosuccinate synthetase; argininosuccinate lyase; arginase; fumarylacetoacetate hydrolase; porphobilinogen deaminase; cystathionine beta-synthase; branched chain ketoacid decarboxylase; isovaleryl-CoA dehydrogenase; propionyl CoA carboxylase; methylmalonyl-CoA mutase; glutaryl CoA dehydrogenase; insulin; pyruvate carboxylase; hepatic phosphorylase; phosphorylase kinase; glycine decarboxylase; H-protein, T-protein, cystic fibrosis transmembrane regulator (CFTR); dystrophin; microdystrophin; CT GaINAc transferase (Galgt2); or survival motor neuron (SMN).

18. A method of making a composition comprising an AAV vector in a LNP, said method comprising:
mixing an AAV vector with a LNP.

19. A method of making a composition comprising an AAV vector in a LNP, said method comprising:
coating a vessel with one or more lipids; and
adding said AAV vector to said lipid coated vessel.

20. A method of making a composition comprising an AAV vector in a LNP, said method comprising:
coating a vessel with said AAV vector; and
adding said LNP or one or more lipids to said AAV coated vessel.

21. The method of any of claims 18-20, wherein said AAV vector or LNP is in a liquid.

22. The method of any of claims 18-20, wherein said AAV vector or LNP comprises an emulsion or is in a solution.

23. The method of claim 21 or 22, wherein said LNP liquid or solution is substantially dried prior to adding said AAV vector or LNP to said coated vessel.

24. A method comprising:
solubilizing one or more lipid components in an organic solvent to provide a lipid solution;
adding said lipid solution to a vessel;
removing said organic solvent;
adding an AAV vector in aqueous solution to said vessel; and
forming LNPs by agitating said vessel by vortexing or sonication.

25. The method of claim 24, wherein the removing step is carried out by heating, vacuum, passing dry air or gas into the vessel or combinations thereof while rotating the vessel.

26. The method of claim 24, further comprising sorting said LNPs to homogeneous size by extrusion through one or more membranes.

27. A method comprising:
solubilizing one or more lipids in an organic solvent;
solubilizing an AAV vector in an aqueous buffer; and
mixing said solubilized lipids and said solubilized AAV vector to form LNPs encapsulating said AAV vector.

28. A method comprising:
solubilizing one or more lipids in an aqueous buffer with a detergent; adding AAV vector to said solubilized lipids; and
removing said detergent by dialysis to form LNPs encapsulating said AAV vector.

29. A method comprising:
solubilizing one or more lipids in an organic solvent; and
adding said solubilized one or more lipids dropwise to an AAV vector in an aqueous buffer with mixing to form LNPs encapsulating said AAV vector.
